(19) United States
(12) Patent Application Publication
(10) Pub. No.: US 2026/0201424 A1
(43) Pub. Date: Jul. 16, 2026

(54) MICROBIAL MEDIATED TRANSMUTATIONS

(71) Applicant: ECOBIOME HOLDINGS, LLC, THE WOODLANDS, TX (US)

(72) Inventor: MARC RODRIGUEZ, THE WOODLANDS, TX (US)

(73) Assignee: ECOBIOME HOLDINGS, LLC, THE WOODLANDS, TX (US)

(21) Appl. No.: 19/135,323

(22) PCT Filed: Dec. 20, 2023
(86) PCT No.: PCT/US2023/085256

§ 371 Date: Jun. 3, 2025

Related U.S. Application Data
(60) Provisional application No. 63/434,358, filed on Dec. 21, 2022.

(51) Int. Cl. C12P 3/00; C12P 5/02; C22B 3/18; C22B 11/00; C22B 26/12; C22B 59/00

(52) U.S. Cl. CPC C12P 3/00; C12P 5/02; C22B 3/18; C22B 11/04; C22B 26/12; C22B 59/00

(57) ABSTRACT

A method of recovering or producing precious metals, platinum group metals, and/or rare earth elements from a starting substrate, the method comprising: contacting the starting substrate with a composition comprising an isolated microbe for an amount of time sufficient for the biological transmutation process to occur, the isolated microbe expressing a first enzyme at least 95% identical to, or at least 98% identical to, or identical to a transmutase enzyme identified by SEQ. ID. NO. 1, and a second enzyme at least 95% identical to, at least 98% identical to, or identical to an amplificase enzyme identified by SEQ. ID. NO. 2. Biological transmutation of silicon to gold. Converting phosphorus in a starting material into phosphoric acid and/or phosphonic acid. Converting heavy oil in a starting material into octane. Methods of extracting, amplifying, or producing lithium from various substrates.

### CROSS-REFERENCE TO RELATED APPLICATIONS

This United States national stage application is entitled to and claims the benefit of earlier filed provisional application No. 63/434,358, filed Dec. 21, 2022, and earlier filed provisional application No. 63/591,270, filed Oct. 18, 2023 under 35 U.S.C. § 119 (e), and claims priority to and is a United States nationalization of Patent Cooperation Treaty application number PCT/US2023/085256, filed Dec. 20, 2023, which earlier filed provisional applications and PCT application are incorporated by reference herein in their entirety.

### BACKGROUND INFORMATION

### Technical Field

The present disclosure is directed to biological systems useful for cold fusion and/or nuclear transmutation. In particular, the present disclosure is directed accelerated methods to transform a chemical and/or element into another chemical or element using microorganisms.

### REFERENCE TO SEQUENCE LISTING

The present application is being filed along with a sequence listing in electronic format. The sequence listing is provided as a file entitled “3414.900.xml” created on Sep. 1, 2023, and which is 4,770 bytes in size. The information in electronic format of the sequence listing is incorporated by reference in its entirety.

### Background Art

In physics, nuclear transmutation is the process of converting one isotope of an element into another chemical element. During this process, the number of neutrons or protons in the nucleus of the resulting element will be different from the original one. Biological transmutation is defined as nuclear transmutation occurring in a living organism (e.g., a microorganism). Generally, the phenomenon is not accepted by mainstream science, which argues that transmutations are only possible in high-energy nuclear reactions and that such reactions are physically impossible in biological systems, as the amount of energy used in such a manner would be fatal within a several-kilometer radius. However, evidence shows that transmutations do occur, and that the lack of a theoretical model adequately explaining the mechanisms involved (that is, without the emission of deadly amounts of energy) does not render that evidence invalid. A French scientist named Corentin Louis Kervran (1901 to 1983) proposed that such nuclear transmutations occur in living organisms too. Kervran investigated discrepancies between the dietary or environmental intake of elements such as calcium, potassium or magnesium by various organisms and the quantities they hold or excrete. For instance, Kervran investigated the source of calcium chickens use for their eggshells and concluded that they probably convert the calcium from dietary potassium.

It would be an advance in the metallurgical and biological arts to find or manufacture mutated or otherwise modified microorganisms that express novel protein enzymes having the ability to promote transmutation of certain elements in a starting substrate to target elements, for example to produce greater amounts of metals than is available from a starting substrate.

### SUMMARY

The compositions and methods of the present disclosure are useful for biological transmutation. Applicant has manufactured or mutated or otherwise modified microorganisms that express novel protein enzymes having the ability to promote transmutation of certain elements in a starting substrate to target elements, for example to produce greater amounts of metals than is available from a starting substrate.

A first aspect of the present disclosure are compositions comprising (or consisting essentially of, or consisting of) an isolated bacterial strain Thiomonas isabelensis (ECOAU001), which has been designated Accession number NRRL No. B-67995, deposited in accordance with the Budapest Treaty at the Agricultural Research Service Culture Collection (USDA, ARS, 1815 North University Street, Peoria, IL, 61064) on Nov. 13, 2020, which express one or both of the transmutase and amplificase enzymes, where the transmutase enzyme is defined by the SEQ. ID. NO. 1, and the amplificase enzyme is defined by SEQ. ID. NO. 2.

| As used herein, SEQ. ID. NO. 1 is: | |
|---|---|
| AVQNESKRYT VSYLKTLNYY DLVDLLAKTE IENLPDLFQY | 60 |
| SSDAKEFYGN KTRMNFIMDE | |
| IGRRASQYTE IDHKGIPTLV EVVRAGFYLG FHNKELNEIN | 120 |
| KRSFKERVIP SILAIQKNPN | |
| FKLGTEVQDK IVSATGLLAG NETSPAEVVN NFTPILQDCI | 180 |
| KNMDRYALDD LKSKALFNVL | |
| AAPTYDVTEY LRATKEKPEN TPWYGKIDGF INEVKKLALY | 240 |
| GKINSRNSWI IDNGIYHIAP | |
| LGKLHSNNKI GIETLTEVMK VYPYLSMQHL QSADQIKRHY | 300 |
| DSKDAEGNKI PLDKFKKEGK | |
| EKYCPKTYTF DDGKVIIKAG ARVEEEKVKR LYWASKEVNS | 360 |
| QFFRVYGIDK PLEEGNPDDI | |
| LTMVIYNSPE EYKLNSVLYG YDTNNGGMYI EPEGTFFTYE | 420 |
| REAQESTYTL EELFRHEYTH | |
| YLQGRYAVPG QWGRTKLYDN DRLTWYEEGG AELFAGSTRT | 480 |
| SGILPRKSIV SNIHNTTRNN | |
| RYKLSDTVHS KYGASFEFYN YACMFMDYMY NKDMGILNKL | 540 |
| NDLAKNNDVD GYDNYIRDLS | |
| SNHALNDKYQ DHMQERIDNY ENLTVPFVAD DYLVRHAYKN | 600 |
| PNEIYSEISE VAKLKDAKSE | |
| VKKSQYFSTF TLRGSYTGGV SKGKLEDQKA MNKFIDDSLK | 660 |
| KLDTYSWSGY KTLTAYFTNY | |
| KVDSSNKVTY DVVFHGYLPN EGDSKNSLPY GKTNGTYKGT | 720 |
| EKEKIKFSSE GSFDPDGKIV | |
| SYEWDFGDGN KSNEENPEHS YDKVGTYTVK LKVTDDKGES | 780 |
| SVSTTTAEIR DLSENKLPVI | |
| YMHVPTSGAL NQKVVFYGKG TYDPDGSIAG YQWDFGDGSD | 840 |
| FSSEQNPSHV YTKKGEYTVT | |
| LRVMDSSGQM SEKTMKIKIT DPVYPIGTEK EPNNSKETAS | 900 |
| GPIVPGIPVS GTIENTSDQD | |
| YFYFDVITPG EVKIDINKLG YGGATWVVYD ENNNAVSYAT | 960 |
| DDGQNLSGKF KADKPGRYYI | |
| HLYMFNGSYM PYRINIEGSV GR | 982 |
| and | |
| SEQ. ID. NO. 2 is: | |
| AVQNESKRYT VSYLKTLNYY DLVDLLAKTE IENLPDLFQY | 60 |
| SSDAKEFYGN KTRMNFIMDE | |
| IGRRASQYTE IDHKGIPTLV EVVRAGFYLG FHNKELNEIN | 120 |
| KRSFKERVIP SILAIQKNPN | |
| FKLGTEVQDK IVSATGLLAG NETSPAEVVN NFTPILQDCI | 180 |
| KNMDRYALDD LKSKALFNVL | |
| AAPTYDVTEY LRATKEKPEN TPWYGKIDGF INEVKKLALY | 240 |
| GKINSRNSWI IDNGIYHIAP | |
| LGKLHSNNKI GIETLTEVMK VYPYLSMQHL QSADQIKRHY | 300 |
| DSKDAEGNKI PLDKFKKEGK | |
| EKYCPKTYTF DDGKVIIKAG ARVEEEKVKR LYWASKEVNS | 360 |
| QFFRVYGIDK PLEEGNPDDI | |
| LTMVIYNSPE EYKLNSVLYG YDTNNGGMYI EPEGTFFTYE | 420 |
| REAQESTYTL EELFRHEYTH | |
| YLQGRYAVPG QWGRTKLYDN DRLTWYEEGG AELFAGSTRT | 480 |
| SGILPRKSIV SNIHNTTRNN | |
| RYKLSDTVHS KYGASFEFYN YACMFMDYMY NKDMGILNKL | 540 |
| NDLAKNNDVD GYDNYIRDLS | |
| SNHALNDKYQ DHMQERIDNY ENLTVPFVAD DYLVRHAYKN | 600 |
| PNEIYSEISE VAKLKDAKSE | |
| VKKSQYFSTF TLRGSYTGGV SKGKLEDQKA MNKFIDDSLK | 660 |
| KLDTYSWSGY KTLTAYFTNY | |
| KVDSSNKVTY DVVFHGYLPN EGDSKNSLPY GKTNGTYKGT | 720 |
| EKEKIKFSSE GSFDPDGKIV | |
| SYEWDFGDGN KSNEENPEHS YDKVGTYTVK LKVTDDKGES | 780 |
| SVSTTTAEIR DLSENKLPVI | |
| YMHVPTSGAL NQKVVFYGKG TYDPDGSIAG YQWDFGDGSD | 840 |
| FSSEQNPSHV YTKKGEYTVT | |
| LRVMDSSGQM SEKTMKIKIT DPVYPIGTEK EPNNSKETAS | 900 |
| GPIVPGIPVS GTIENTSDQD | |
| YFYFDVITPG EVKIDINKLG YGGATWVVYD ENNNAVSYAT | 960 |
| DDGQNLSGKF KADKPGRYYI | |
| HLYMFNGSYM PYRINIEGSV GR | 982 |

Through various characterization methods it was found that the microorganism facilitated precious metal, rare earth, and gold production from a variety of environmental solid and liquid substrata. The isolated microorganism demonstrated gold and precious metals production de novo, using eco-friendly and sustainable biochemical processes and methods. Altogether, the present disclosure provides multiple lines of evidence showing microorganisms useful to extract, produce and/or amplify precious metals and/or rare earth metals from a variety of environmental substrates. Other aspects and iterations of the invention are described more thoroughly below.

### BRIEF DESCRIPTION OF THE DRAWINGS

The manner in which the objectives of this disclosure and other desirable characteristics can be obtained is explained in the following description and attached drawings in which:

FIG. 1 shows the amino acid sequence of the transmutase enzyme, SEQ. ID. NO. 1;

FIG. 2 shows the amino acid sequence of the amplificase enzyme, SEQ. ID. NO. 2;

FIGS. 3, 4, 5, 6, 7, and 8 present example data in graphical and tabular form; and

FIG. 9 illustrates conceptually some of the methods of the present disclosure.

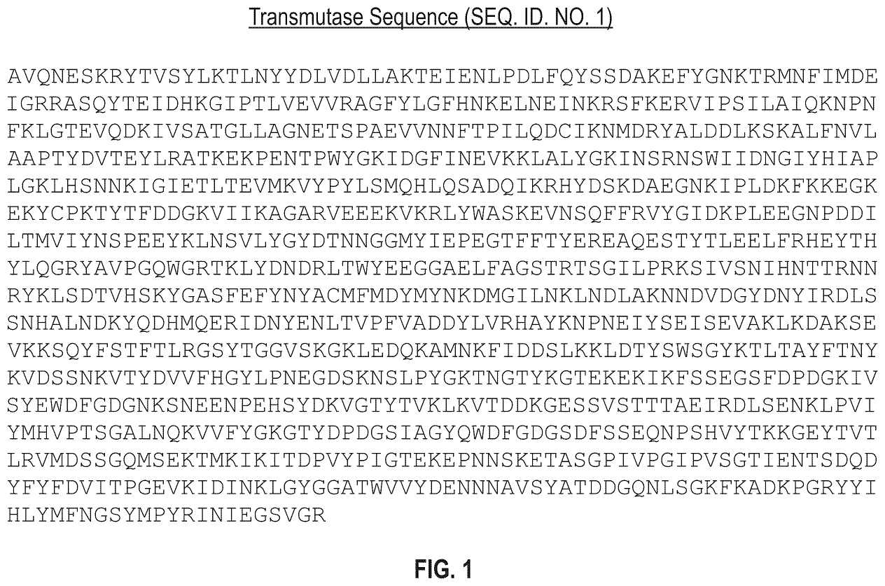

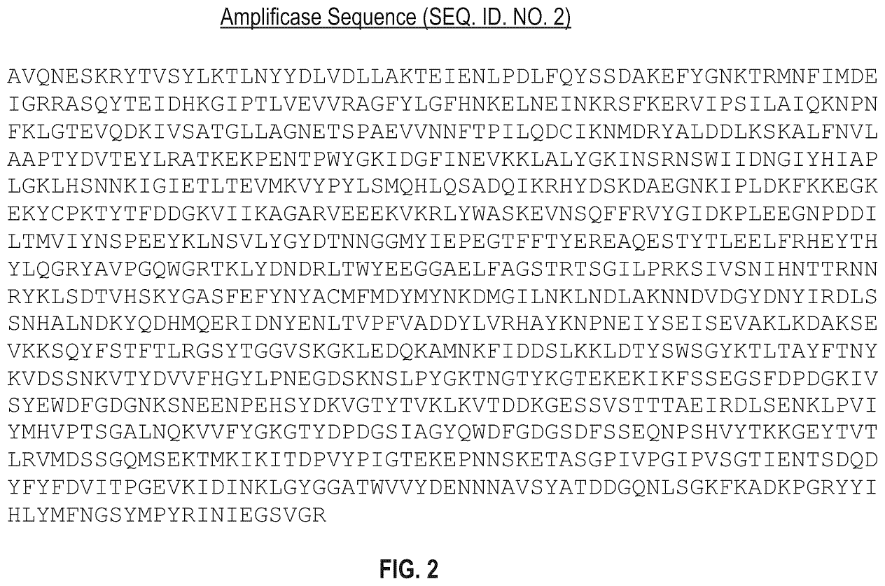

It is to be noted, however, that the appended drawings may not be to scale and illustrate only typical embodiments of this disclosure. Therefore, the drawing figures are not to be considered limiting in scope, for the disclosure may admit to other equally effective embodiments.

### DETAILED DESCRIPTION

In the following description, numerous details are set forth to provide an understanding of the compositions and methods of the present disclosure. However, it will be understood by those skilled in the art that the compositions and methods disclosed herein may be practiced without these details and that numerous variations or modifications from the described embodiments may be possible. All technical articles, published and non-published patent applications, standards, patents, statutes, and regulations referenced herein are hereby explicitly incorporated herein by reference, irrespective of the page, paragraph, or section in which they are referenced. Where a range of values describes a parameter, all sub-ranges, point values and endpoints within that range or defining a range are explicitly disclosed herein. All percentages herein are by weight unless otherwise noted.

All numbers disclosed herein are approximate values, regardless whether the word “about” or “approximate” is used in connection therewith. They may vary by 1%, 2%, 5%, and sometimes, 10 to 20%. Whenever a numerical range with a lower limit, RL and an upper limit, RU, is disclosed, any number falling within the range is specifically disclosed. In particular, the following numbers within the range are specifically disclosed: R=RL+k*(RU−RL), wherein k is a variable ranging from 1% to 100% with a 1% increment, i.e., k is 1%, 2%, 3%, 4%, 5%, . . . , 50%, 51%, 52%, . . . , 95%, 96%, 97%, 98%, 99%, or 100%. Moreover, any numerical range defined by two R numbers as defined in the above is also specifically disclosed.

It should be understood that wherever the term “comprising” is used herein, other embodiments where the term “comprising” is substituted with “consisting essentially of” are explicitly disclosed herein, and vice versa. It should be further understood that wherever the term “comprising” is used herein, other embodiments where the term “comprising” is substituted with “consisting of” are explicitly disclosed herein, and vice versa. Moreover, the use of negative limitations is specifically contemplated. The term “comprising” and derivatives thereof is not intended to exclude the presence of any additional component, step or procedure, whether or not the same is disclosed herein. In order to avoid any doubt, all compositions and methods claimed herein through use of the term “comprising” may include any additional component, step, additive, adjuvant, or compound whether monomeric, oligomeric, polymeric or otherwise, unless stated to the contrary. In contrast, the term, “consisting essentially of” excludes from the scope of any succeeding recitation any other component, step or procedure, excepting those that are not essential to operability. The term “consisting of” excludes any component, step or procedure not specifically delineated or listed. The term “or”, unless stated otherwise, refers to the listed members individually as well as in any combination. The term “a” includes a single item as well as multiple items.

Applicant has discovered microbes with the ability to express certain protein enzymes (referred to herein as the transmutase enzyme (SEQ. ID. No. 1) and the amplificase enzyme (SEQ. ID. NO. 2)) that promote the recovery of precious metals, platinum group metals and rare earth elements from different substrates. The inventor herein contemplates that other isolated microbes expressing a first enzyme at least 95% identical to, or at least 98% identical to, or identical to a transmutase enzyme identified by SEQ. ID. NO. 1, and a second enzyme at least 95% identical to, at least 98% identical to, or identical to an amplificase enzyme identified by SEQ. ID. NO. 2 may be used in the methods of the present disclosure. The enzymes are not separated from the microbes prior to their use in methods of the present disclosure but exist within the live microbes. The microbes were isolated using an apparatus known under the trade designation EcoBiome™, the apparatus being described in assignee's co-pending application Ser. No. 17/895,616, filed Aug. 25, 2022, whose parent application Ser. No. 16/258,112, filed Jan. 25, 2019, was published as US20190232276A1, Aug. 1, 2019; the '616 and '112 applications and the '276 published application are incorporated by reference herein in their entireties. For example, Applicant discovered that the isolated microbes, modified according to the methods of the present disclosure, and their expressed transmutase and/or amplificase enzymes, have the unparalleled ability to produce greater amounts of metals than is detectable in the starting substrate well beyond current known technology and methods.

As one unlimiting example, Applicant has shown the ability to produce a higher concentration of the rare earth element, neodymium, from what was found in the rock ore substrate. In one example, the beginning rock ore substrate consisted of 0.01%/wt. neodymium metal with other metals throughout the ore matrix. The final concentration of neodymium, after biological transmutation using a microbe of the present disclosure, unexpectedly resulted in a 0.15%/wt. neodymium concentration, an increase of more than one order of magnitude.

In another exemplary embodiment, silicon is transmutated directly into gold by contacting a starting substrate comprising silicon with an isolated microbe of the disclosure for an amount of time sufficient for the biological transmutation process to occur. This method bypassed the quartz stage (Silicon Dioxide) through a novel form of energy conversion.

Thus, the present disclosure provides the uses of microbes for recovering precious metals from a starting material (e.g., mineral ores). Precious metals are frequently occluded, encapsulated, bonded and/or alloyed in mineral ores and are not amendable to conventional recovery methods. For example, gold often occurs as finely disseminated submicroscopic particles within a refractory sulfide host of pyrite or arsenopyrite. Prior to the compositions and methods of the present disclosure, bio-oxidation was used to liberate the gold occluded within the sulfide host. A few processes for bio-oxidizing metal sulfide minerals are known in the art. One known method of bio-oxidizing the metal sulfides in an ore is to use bacteria, such as Thiobacillus ferrooxidans, sulfolobus, acidianus species and facultative-thermophilic bacteria in a microbial pretreatment. In contrast, the compositions and methods of the present disclosure are capable of producing a greater mass of metals through biological activity than what is currently possible by conventional methods using conventional recovery methods.

In another exemplary embodiment, a composition and method of the present disclosure provides the rapid conversion of aluminum in a starting substrate into the rare earth element neodymium by contacting the starting substrate with an isolated microbe for an amount of time sufficient for the biological transmutation process to occur.

According to another exemplary embodiment, a composition and method of the present disclosure provides the conversion of phosphorus into phosphoric acid and phosphonic acid by contacting a substrate with an isolated microbe of the present disclosure for an amount of time sufficient for a biological transmutation process to occur. For example, the present disclosure provides the use of seawater components to convert indigenous phosphorus in seawater (0.003%/wt.) to phosphonic acid (0.025%/wt.) and phosphonate acid (0.048%/wt.) through microbial processing.

According to another exemplary embodiment, a composition and method of the present disclosure provides the conversion of spent/waste heavy oil into octane by contacting a substrate with an isolated microbe of the disclosure for an amount of time sufficient for a biological transmutation process to occur. For example, the present disclosure provides the use spent/waste heavy oil (0.15%/wt.) into a new octane hydrocarbon (0.75%/wt.) through transmutation of the long chain hydrocarbon solubilized in sea water and bacteria.

According to yet another exemplary embodiment, a composition and method of the present disclosure provides for increased yttrium production by contacting a substrate with an isolated microbe of the present disclosure for an amount of time sufficient for a biological transmutation process to occur. For example, the disclosure provides increased production of yttrium rare earth from a concentration of 0.0017%/wt. in a waste substrate of coal into a higher concentration and yield of yttrium (0.099%/wt.) after bacterial transmutation.

According to still another exemplary embodiment, a composition and method of the present disclosure provides the conversion of silicon (0.002%/wt.) found in farm soil into gold (0.55%/wt.) through bacterial transmutation.

According to one embodiment, the microbes used herein are single-celled and non-pathogenic. In one aspect of the present disclosure encompasses an isolated bacteria strain Thiomonas isabelensis (ECOAU001), which has been designated Accession number NRRL No. B-67995, deposited in accordance with the Budapest Treaty at the Agricultural Research Service Culture Collection (USDA, ARS, 1815 North University Street, Peoria, IL, 61064) on Nov. 13, 2020. ECOAU001 is particularly efficient for the biological transmutation process. Without being bound to any particular theory, I believe this is because the modified or mutated form of T. isabelensis (ECOAU001) we have achieved as explained in the examples herein, including their expressed transmutase enzyme (SEQ. ID. NO. 1) and/or amplificase enzyme (SEQ. ID. NO. 2), have the unique and unexpected ability to transmute elements as described herein. Certain compositions of the present disclosure may include other known microbes (modified or unmodified, mutated or non-mutated) as long as there are present the requisite amount of Thiomonas isabelensis (ECOAU001) and at least one of their expressed transferase and/or amplificase enzymes to perform the desired transmutation. Other microbes and their expressed enzymes that may be present include, but are not limited to, Aspergillus niger, Aspergillus orzae, Ashbya gossypii, Streptomyces species, Bacillus thuringiensis, Rhizobium, Bradyrhizobium, Bacillus subtilis, Corynebacterium glutamicum, Leuconostoc mesenteroides, Streptodornase pyogenes, and Thiobacillus ferrooxidans. Alternatively, in certain embodiments, one or more of these known microbes may be mutated to confer the ability of the mutated versions to express the transmutase enzyme (SEQ. ID. No. 1) and/or amplificase enzyme (SEQ. ID. NO. 2) and carry out the biological transmutation process of ECOAU001.

In accordance with the present disclosure compositions comprising microorganisms and enzymes of the present disclosure for use within the methods of the disclosure may comprise one or more additional components for various functions. Such additional components may include, but are not limited to, biosolvents, ethyl lactate, ATP, ADP, pyrophosphate, soy based solvents, chemical solvents, green solvents, one or more organic acids such as (but not limited to) lactic acid, malic acid, ascorbic acid, alkanes, alkenes, alkynes, saturates, aromatics, resinoids, asphaltenes, light, mid chain and heavy chain hydrocarbons, sodium nitrate, sodium nitrite, ethanol, sulfur, sulfate, sulfite, nitrogen, chemical surfactants (ionic, anionic, cationic, zwitterionic surfactants), polymers (low, mid, heavy Chains), biosurfactants, glycolipids, rhamnolipids (J1 and J2), glycerin, propylene glycol, carbon sugars, dextrose, galactose, sucrose, fructose, complex carbohydrates, starch, cellulose, lignin, keratin, proteins and amino acids, fertilizer NPK (e.g., organic and inorganic fertilizers), manures, composts, green waste, sludge material, humic and fulvic acids, coal ash and coal derived waste, alumina cytokinins and seaweed extracts.

According to the present disclosure compositions comprising microorganisms of the disclosure for use within the methods of the disclosure may comprise a water source for microbial culturing or final product carrier. Non-limiting examples include deionized water, distilled water, filtered water, well water, tap water, fresh water, sea water, brackish water, mineralized water, carbonated water, saline water, ionically charged water, ionized water, and hydrogen water. The aqueous solution may contain sufficient nutrients to support microbial growth. The useful nutrients are both inorganic and organic compounds commonly used to grow and nourish microbes. Inorganic nutrients include nitric acid, ammonium nitrate, ammonium chloride, ammonium sulfate, sodium nitrate, sulfur, sodium sulfide, sodium chloride, sodium bicarbonate, sodium phosphate, potassium phosphate, sulfuric acid, nitric acid, cyanide, uranium, mercury, lead, lithium, sodium metabisulfite, ammonium nitrate, fertilizers, gluconic acid, phosphogypsum, ferric chloride, calcium chloride, and ammonium phosphate. Organic nutrients include microbial biomass, glucose, dextrose, sodium acetate, amino acids, and purines. Vitamins that can be included in the nutrient solution include pyridoxine, pyridoxamine-HCl, riboflavin, thiamine, niacin, pantothenic acid, p-aminobenzoic acid, folic acid, and biotin. Small amounts of trace elements such as iron, copper, molybdenum and zinc can also be provided in the nutrient solution. Useful nutrients can also be mineral ores used for recovery of metals.

According to the present disclosure compositions comprising microorganisms of the disclosure for use within the methods of the disclosure may be formulated as a soil mixture, liquid, sludge or slurry substrate.

In some embodiments, the biological transmutation process of producing precious metals and/or rare earth metals generally comprises farming the precious metals and/or rare earth metals including the steps of inoculating microbes on solid substrates or geological substrates. A geologic substrate is a surface (or volume) of sediment or rock where physical, chemical, and biological processes occur, such as the movement and deposition of sediment, the formation of bedforms, and the attachment, burrowing, feeding, reproduction, and sheltering of organisms. Non-limiting examples of a geological substrate useful for methods of the present disclosure include sandstone, limestone, shale, coal, chalk deposit formations, refractory rock ore (e.g., single, double and triple refractory rock ore). Additional solid substrates include, but are not limited to an environmental sample collected from any terrestrial, aquatic or marine source such as soil, biofilms, sediments (e.g. coral or other marine sediments, aquifer sediments and the like), native metal rocks and sludge residue. In some embodiments, the solid substrate may be disinfected prior to inoculation of the microbes of the disclosure. Disinfection techniques include but are not limited to steam, auto-clave, oven, microwave, biocide and fungicide solutions. Additional starting substrates include but are not limited to animal manures, bauxite, base metals, calcium phosphate, calcium silicate, clays and silicates, aluminum oxide, diatomaceous earth, diammonium phosphate, erionite and other zeolites, feldspar, flint, food wastes, granite, graphite, gypsum, humic and fulvic acids, marble, mica, molten rock and lava, monoammonium phosphate, potash, pumice, silica, slate, seaweed, talc and recycled electronics and commercial devices.

Indeed the starting substrate is not particularly limited, and only requires the presence of the specific element to be converted.

In some embodiments, microbes are applied to any solid substrate in a rock, powder, granulated or broken form for improved precious metals, platinum metals and rare earth metal leaching and extraction. In some embodiments, solid substrates are used in traditional farming, specialty farming, potted and greenhouse farming, hydroponics and aeroponics techniques.

For purposes of this disclosure, the term “mineral” or “mineral ore” means a composition that comprises precious metal values. Thus, a mineral may be a mined mineral, ancient seabed deposit, ancient lakebed deposit, black sands, an ore concentrate, metal bearing sea water, and waste products, such as mining tails, industrial waste water, oil well brine, coal tars, oil shales, tar sands, and oil sands. Useful minerals contain trace amounts of precious metals. Trace amount means the detection limit or below detection limits of conventional assay procedures such as fire assay, AAS (atomic adsorption spectroscopy), ICP-MS (inductively coupled plasma-mass spectrometer), ICP-AES (inductively coupled plasma-atomic emission spectroscopy) and other spectroscopic instrumentation commonly used in analytical laboratories. Some spectroscopic methods can detect as little as 1 ppt (part per trillion) to 0.1 ppb (part per billion).

As used herein, the term “rare earth metals” or “RE” may refer to scandium (Sc), yttrium (Y), lanthanum (La), cerium (Ce), praseodymium (Pr), neodymium (Nd), promethium (Pm), samarium (Sm), europium (Eu), gadolinium (Gd), terbium (Tb), dysprosium (Dy), holmium (Ho), erbium (Er), thulium (Tm), ytterbium (Yb), and/or lutetium (Lu). As used herein, the term “precious earth metals” may refer to gold, silver, aluminum, rhenium, indium, platinum, gallium, germanium, ruthenium, rhodium, beryllium, palladium, osmium, iridium, tellurium, bismuth, platinum palladium, titanium, zinc, and zirconium.

In one embodiment, the biological transmutation process using microbes according to the disclosure may be conducted in a commercially available bioreactor with agitation. The agitation may be accomplished by mechanical stirring with a flat bladed impeller, percolation column, or air agitated pachuca reactor. The bioreactor may have one or more air intakes, sterilization members, harvesting passages, heating and/or cooling facilities, a temperature controller, a pH controller, one or more filters, and a pressure controller. All these features of bioreactors are known and commercially available in the biotechnology industry.

The biological transmutation processes using microbes according to the present disclosure may also be performed by heap leaching techniques. In heap bio leaching techniques, a large body of mineral ore is treated with mutant microbes in nutrient solution in large contaminant ponds with no agitation and/or only occasional agitation. Generally, the contact time for heap type bio treatment is substantially longer than the agitated bioreactors and may range from about 10 days to about 100 days.

Bio treatment temperature may range from about 15 degrees centigrade to about 50 degrees centigrade, or from about 20 degrees to about 30 degrees centigrade. pH can be acidic (with pH ranging from about 1 to about 3) or basic (pH ranging from about 9 to 12), although slightly acidic (pH 4) to slightly basic (pH 8) pH ranges are preferred. The most preferred pH ranges are the neutral range of from pH 6.5 to pH 7.5.

In accordance with the methods of the present disclosure, pressure is not critical and can be at atmospheric, below atmospheric, and/or above atmospheric. The biological transmutation process can be conducted in aerobic or anaerobic conditions. The biological transmutation process can be conducted in the presence of nitrogen, carbon dioxide, and oxygen in the atmosphere. Oxygen can be provided chemically, for example, with hydrogen peroxide, or as a gas from pressurized vessels.

Microbe concentration is not critical. At low microbe concentration, the contact duration is generally longer to allow the microbe to grow and multiply. However, microbe concentration should not exceed the maximum microbe concentration that the nutrient solution can sustain. Contact time can vary from a few hours to several weeks and depends in part on the type and mesh size of the mineral ore digested. Contact time ranges can be from 1 day to 30 days, more preferably from 1 day to 10 days.

The biological transmutation methods of using microbes according to the present disclosure can be conducted in aerobic or anaerobic conditions. However, contacting of microbes with substrates is preferably conducted in the presence of oxygen, nitrogen and carbon dioxide in the atmosphere. Oxygen can also be provided chemically, for example, with hydrogen peroxide, or as a gas from pressurized vessels.

Nutrients can also be provided during the biological transmutation process to support growth of the mutant microbes. Nutrients can be inorganic, including nitric acid, sulfur, ammonium nitrate, ammonium chloride, ammonium sulfate, sodium nitrate, sodium chloride, sodium bicarbonate, sodium phosphate, potassium nitrate, potassium phosphate, ferric chloride, calcium chloride, and ammonium phosphate, and organic, including glucose, dextrose, sodium acetate, amino acids, and purines. Vitamins that can be included in the nutrient solution include pyridoxine, pyridoxamine-HCl, riboflavin, thiamine, niacin, pantothenic acid, p-aminobenzoic acid, folic acid, and biotin. Small amounts of traces elements such as iron, copper, molybdenum and zinc can also be provided in the nutrient solution.

In a still another aspect, biological transmutation methods of the present disclosure using microbes according to the disclosure occur in liquid substrates. Suitable liquid substrates include but are not limited to balanced salt and nutrient solutions, broths, environmental samples collected from any aquatic or marine source, waste waters, sludge waters, saltwater, freshwater, irrigation systems, ponds, lakes, rivers, and estuaries. In some embodiments, the liquid substrate is disinfected prior to inoculation of the bacterial strains disclosed herein.

Inoculation of the liquid substrate can occur by any means known to the skilled artisan at concentration described above for the solid substrate. The inoculating step can occur one or more times during the duration of extracting, producing and/or amplifying precious metals and/or rare earth metals from the liquid substrate. After inoculation the liquid substrate is preferably agitated during the extraction.

After inoculation, optionally specialty nutrients and by-products can be added to the inoculated substrate one or more times to establish new, increased and rigorous colonization by the bacterial strain, for example by adding solutions comprising a complex or simple sugar, a seaweed or cytokinin and a vitamin blend. In addition, for accelerated reactions it may be useful to establish an anodic and cathodic LED using a wavelength generator. In certain embodiments, the wavelength generator may generate wavelengths ranging from about 2.0 to about 200 KHz, or from about 2.0 to about 22 KHz, or from about 100 to about 200 KHz.

In each of the above embodiments, a bioreactor, fermenter, reaction vessel can be used in the disclosed methods. Moreover, the present disclosure contemplates the use of the disclosed microbes for bioleaching and heap leaching and therefore the use of leach pits are contemplated within the methods as well.

After the biological transmutation process, the recovery of metal produced from the starting material and microbial solution can be performed by conventional metallurgical methods such as smelting, leaching, electrolysis, resins and other methods known to those skilled in art of metallurgy. In another embodiment, the precious metals in the microbes or biomass of dead microbes can be recovered by methods described for recovery of precious metals from mineral ore.

Fire assaying and cupellation are described by C. W. Ammen, Recovery and Refining of Precious Metals, second edition 1993, Chapter 12, pp 302-329.

The disclosed methodology and compositions using biological transmutation can be easily implemented, is robust, and can be used in the development of various industrial applications, such as but not limited to: harvesting and production of precious metals, platinum group metals, and/or rare earth metals; production and/or recovery (recycling) of elements within the cement industry; production and/or recovery (recycling) of elements related to silicone chips and/or silicone metal; production and/or recovery (recycling) of elements related to semiconductors; production and/or recovery (recycling) of elements related to micro-processing chip and computers; production and/or recovery (recycling) of elements within the chemical production industry; production and/or recovery (recycling) of elements within the automotive industry; production and/or recovery (recycling) of elements within the plastics industry; production and/or recovery (recycling) of elements within the Hydrocarbons/Oil & Gas industry; production and/or recovery (recycling) of elements within the Magnets industry; production and/or recovery (recycling) of elements within the Electric batteries industry; production and/or recovery (recycling) of elements within the Quantum processing equipment and computing; production and/or recovery (recycling) of elements within the Electric batteries and battery production industry; production and/or recovery (recycling) of elements within the Cell phone and telecommunications industry; production and/or recovery (recycling) of elements within agriculture to include horticulture, turf, arbor-ology, soil science and ornamental industries; production and/or recovery (recycling) of elements within the Photovoltaic industry; production and/or recovery (recycling) of elements within the Clean and renewable industry; production and/or recovery (recycling) of elements within the Pharmaceutical industry; production and/or recovery (recycling) of elements within the Laboratory instruments and labware; and production and/or recovery (recycling) of elements within the Mining industry.

### General Techniques

The practice of the present disclosure will employ, unless otherwise indicated, conventional techniques of molecular biology (including recombinant techniques), microbiology, cell biology, biochemistry, and immunology, which are within the skill of the art. Such techniques are explained fully in the literature, such as Molecular Cloning: A Laboratory Manual, second edition (Sambrook, et al., 1989) Cold Spring Harbor Press; Oligonucleotide Synthesis (M. J. Gait, ed. 1984); Methods in Molecular Biology, Humana Press; Cell Biology: A Laboratory Notebook (J. E. Cellis, ed., 1989) Academic Press; Animal Cell Culture (R. I. Freshney, ed. 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds. 1993-8) J. Wiley and Sons; Methods in Enzymology (Academic Press, Inc.); Handbook of Experimental Immunology (D. M. Weir and C. C. Blackwell, eds.): Gene Transfer Vectors for Mammalian Cells (J. M. Miller and M. P. Calos, eds., 1987); Current Protocols in Molecular Biology (F. M. Ausubel, et al. eds. 1987); PCR: The Polymerase Chain Reaction, (Mullis, et al., eds. 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practice approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds. Harwood Academic Publishers, 1995); DNA Cloning: A practical Approach, Volumes I and II (D. N. Glover ed. 1985); Nucleic Acid Hybridization (B. D. Hames & S. J. Higgins eds. (1985»; Transcription and Translation (B. D. Hames & S. J. Higgins, eds. (1984»; Animal Cell Culture (R. I. Freshney, ed. (1986»; Immobilized Cells and Enzymes (IRL Press, (1986»; and B. Perbal, A practical Guide To Molecular Cloning (1984); F. M. Ausubel et al. (eds.).

So that the present disclosure may be more readily understood, certain terms are defined. Unless defined otherwise, all technical and scientific terms used herein have the same meaning as commonly understood by one of ordinary skill in the art to which embodiments of the invention pertain. Many methods and materials similar, modified, or equivalent to those described herein can be used in the practice of the embodiments of the present invention without undue experimentation, the preferred materials and methods are described herein. In describing and claiming the embodiments of the present invention, the following terminology will be used in accordance with the definitions set out below.

The term “about,” as used herein, refers to variation of in the numerical quantity that can occur, for example, through typical measuring techniques and equipment, with respect to any quantifiable variable, including, but not limited to, mass, volume, time, distance, and amount. Further, given solid and liquid handling procedures used in the real world, there is certain inadvertent error and variation that is likely through differences in the manufacture, source, or purity of the ingredients used to make the compositions or carry out the methods and the like. The term “about” also encompasses these variations, which can be up to ±5%, but can also be ±4%, 3%, 2%, 1%, etc. Whether or not modified by the term “about,” the claims include equivalents to the quantities.

When introducing elements of the present disclosure or the preferred aspects(s) thereof, the articles “a,” “an,” “the,” and “said” are intended to mean that there are one or more of the elements. The terms “comprising,” “including,” and “having” are intended to be inclusive and mean that there may be additional elements other than the listed elements.

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

As various changes could be made in the above-described materials and methods without departing from the scope of the invention, it is intended that all matter contained in the above description and in the examples given below, shall be interpreted as illustrative and not in a limiting sense.

### EXAMPLES

The following examples are included to demonstrate various embodiments of the present disclosure. It should be appreciated by those of skill in the art that the techniques disclosed in the examples that follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1: Novel Microorganism Isolation and Classification

It has been estimated that only 2% of all microbial isolates can be cultured in a lab. Therefore, the microbes that can be grown in the laboratory represent only a small fraction of the total diversity that exists in nature. At all levels of microbial phylogeny, uncultured clades that do not grow on standard media are playing critical roles in cycling carbon, nitrogen, and other elements, synthesizing novel by-products, and impacting the surrounding organisms and environment. The ability to culture difficult to culture or previously uncultured microbial strains provides a wealth of information about their role in the environment, ecology, and nutrient cycling. But perhaps even more importantly, screening of novel isolates will reveal novel products that can have profound effects for the discovery of novel drugs, improve agricultural techniques and products, and in the production of rare and precious metals. To solve this problem the inventor has developed a novel apparatus (Ecobiome™ Discovery apparatus) allowing for the controlled growth, isolation and characterization of microorganisms including those that are difficult to culture or are uncultivable at the present time.

In an effort to isolate and characterize novel microbes, an experiment utilizing the apparatus known under the trade designation Ecobiome™ Discovery apparatus was used. A rare earth mining sample originating from near Austin, TX was obtained and prepared for culturing in the apparatus known under the trade designation Ecobiome™ Discovery apparatus. In short, the rare earth mining sample was crushed and loaded into the apparatus known under the trade designation Ecobiome™ for microbial gradiation and speciation. The prepped rare earth mining sample was allowed to equilibrate and microbial growth promoted. After some time a population of microorganisms, including novel organism (ECOAU1), were isolated.

After isolation of the microbe population comprising ECOAU1, challenge tests were performed, which included tests specific for precious and rare metal remediation microbes. These tests resulted in the isolation and characterization of ECOAU1 (Thiomonas isabelensis).

ECOAU1 is a gram-negative bacterium, non-spore former that is capable of metabolizing simple and complex polymers as well as metals through heterotrophic and chemoheterotrophic biochemical pathways. ECOAU1 is a facultative anaerobe and is tolerant of low and high temperatures, e.g., ranging between about 5° C. to about 46° C. In addition, ECOAU1 is tolerant of a pH range from about 2 to about 9. The microorganism reaches exponential growth with high agitation (>360 rpm) and oxygenation (DO>90%) within 6-8 hours.

### Example 2: Manufacturing Precious and Rare Earth Elements

Ten pounds of soil taken from a North Carolina riverbed was used as the processing soil for the trial. The ten pounds were weighed out and placed in a bucket container fashioned specifically for holding soil and dry material during processing in a bioreactor.

The bucket of soil was inserted into the bioreactor and the reactor was filled to 40 gallons using filtered water. Once the water level exceeded the height of the bucket the circulating water pump was turned on in addition to a continuous air pump into the bioreactor.

Prior to inoculation with the microorganism, a disinfection process was initiated using Calcium Hypochlorite followed by Ascorbic Acid neutralization. This allowed for the disinfection of any contaminant microorganisms that may have been present in water, soil or air contaminants to be removed.

After ascorbic acid neutralization of the bioreactor contents, the EcoBiome AU001 microorganism, labeled Thiomonas isabelensis (ECOAU001) was transferred and inoculated into the bioreactor at a rate of 1.0×10¹⁰ CFU/ml.

Nutrient contents were then added which consisted of a blend of NPK fertilizer, seaweed extract, zinc citrate, zinc sulfate, Vitamin blend, Dextrose and 3 liters of Nutrient Broth (autoclaved and sterilized).

The reaction was closed and sealed and allowed to react for 48 hours with an internal liquid temperature of approximately 40° C. using continuous aeration and recirculation.

Once the reaction was completed, all liquid contents were run through centrifugation, harvested, dried in an oven and then analyzed via an X-Ray Fluorescence Analyzer for precious metals and rare earth elements. % Gold production is reported in Table 1.

TABLE 1
EcoAU001 gold concentrations

| | Gold (Au) % by Weight |
|---|---|
| Bioreactor Trial | (X Ray Fluorescence Analyzer) |
| Untreated Control | 0 (Less than the |
| | Limit of Detection) |
| Sample 1 - Centrifugation 1x | 0.369 |
| Sample 2 - Centrifugation 2x | 0.984 |
| Sample 3 - Soil attached to | 3.676 |
| bucket screen | |

Average % by Weight extraction of Gold (Au) is in the range of 10 grams-15 grams per 2,000 pounds of rock and soil (0.000011%-0.000016% extraction efficiency rate). All (3) Trial samples significantly increased extraction efficiencies compared to the Untreated control soil.

As can be seen in Table 1, the untreated control (soil without ECOAU001 inoculation) lacked a gold concentration within the limit of detection by the analyzer. Sample 1 was completed and was run through the centrifuge at 8000 rpm 1 time. Sample 2 was the effluent after sample 1 was centrifuged and then run through the centrifuge a 2^{nd} time, to determine whether more material could be captured by the centrifuge. Sample 3 was located at the screens we placed to hold the solid soil substrate. A layer of soil like material being captured on the screen throughout the process was identified. Therefore, we decided to take a sample for testing and called this the bucket screen sample.

### Example 3: Microbial Gold and Precious Metals Farming (Includes Traditional Farming, Specialty Farming, Potted and Greenhouse Farming, Hydroponics and Aeroponics)—Soil, Native Metal Rocks and Geological Substrates

The starting soil was disinfected with a standard biocide and fungicide to reduce microbial colony and propagule concentrations to below or about 5.0×10⁵ CFU/Gm.

The soil was then inoculated using a soil drench, irrigation, or overhead irrigation treatment with ECOAU001 plus ECOAU001 nutrients and by-products at a minimum concentration CFU/Gm soil of about 1.0×10⁶ CFU/gm-1.0×10¹² CFU/Gm.

The soil was then tilled up to 4-6 inches to allow for greater surface area contact between the ECOAU001 microbes and the soil.

The soil was lightly irrigated and fertilized with a low NPK plus micronutrient fertilizer for stimulating ECOAU001 microbial colonization and exponential growth throughout the soil or substrate.

The treated soil was then covered with a tarp or cover to maintain a stable temperature or allow for an increase in the soil temperature to a range between about 29° C.-50° C.

The treated soil was lightly irrigated weekly or more frequently in some areas to improve colonization concentration and growth.

Post primary inoculation one or more additional inoculations of ECOAU001 plus specialty nutrients and by-products occurred every 2-4 weeks to re-establish new, increased and rigorous colonization by the microbes.

During the gold and precious metal production phase one or more tests were performed to confirm ECOAU001 mediated gold and precious metals production and extraction through standard precious metals testing and analysis.

Harvest the precious metals and gold occurred by removing the top 2-4 inches of soil, transferring to a contained area with a floor lining, adding water to the soil and bringing the mixture to a slurry. The slurry is then centrifuged through continuous centrifugation at a minimum of 8,000 RPM to concentrate the precipitate which includes contains the de novo gold and precious metals. This process was repeated thereby enriching for the de novo gold and precious metals.

### Example 4: Microbial and Precious Metals Extraction-Liquid Solutions and Substrates (Saltwater, Freshwater, Irrigation Systems, Ponds, Lakes, Rivers, Estuaries And any Body of Water)

The starting water or starting liquid solution were disinfected with a standard biocide to reduce microbial colony and propagule concentrations to below or at about 5.0×10⁵ CFU/ml.

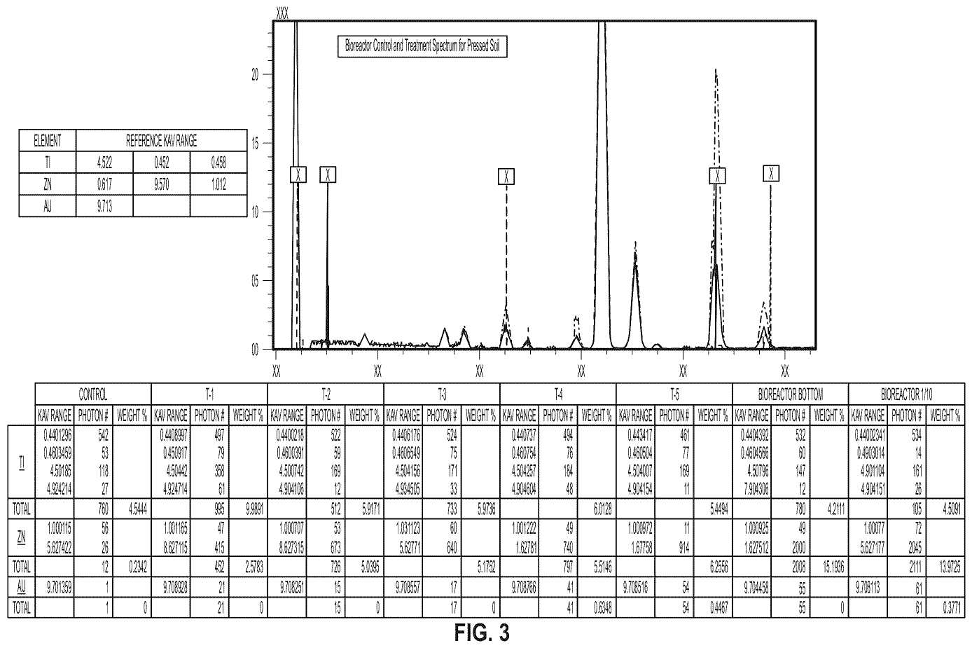

Freshwater, brackish and seawater samples were inoculated with ECOAU001 plus ECOAU001 nutrients and by-products at a minimum concentration CFU/ml liquid of about 1.0×10⁶ CFU/ml-1.0×10¹² CFU/ml. The inoculated water samples were then agitated using an air pump for aerobic respiration. For accelerated reactions an anodic and cathodic LED was used using a wavelength generator set at a range of 2.0-22.0 KHz. The inoculated water samples were then allowed to culture and produce precious metals for 24-48 hours. To harvest the precious metals, the liquid solution was centrifuged through an in line and continuous centrifuge at a minimum of 8,000 RPM to concentrate the metal precipitation. Results are present in FIG. 3.

### Example 5: EcoBiome Analysis of Fire Assay samples

The goal of this analysis is to determine whether EcoBiome's Rare Metals Division proprietary treatment produces a gold (Au) recovery rate statistically higher than the sample control content of 4.19 ppm. A sample of about 12 pounds from a gold mine was treated. Treated samples were sent to Hazen Research, Inc lab to measure Au content in ppm by the fire assay method. In this example the following 10 samples were analyzed. Results are detailed in Table 2.

TABLE 2
Gold Recovery Rate
Sample Results

| | | | Gold in | Gold in |
|---|---|---|---|---|
| Sample ID | Treatment | Batch | ppm | g/ton |
| 21M01497-001 | Control | | 4.22 | 3.83 |
| 21M01497-002 | Centrifuge | | 3.39 | 3.08 |
| 21M01585-001 | Centrifuge | Mar. 8, 2021 | 10.30 | 9.34 |
| 21M01585-002 | Centrifuge | Mar. 8, 2021 | 7.68 | 6.97 |
| 21M01585-004 | Centrifuge | Mar. 22, 2021 b | 7.54 | 6.84 |
| 21M01585-006 | Control | | 4.18 | 3.79 |
| 21M01718-003 | Centrifuge | Apr. 5, 2021 (1) | 13.10 | 11.90 |
| 21M01718-004 | Centrifuge | Apr. 5, 2021 (2) | 10.70 | 9.71 |
| 21M01718-017 | Centrifuge | Apr. 12, 2021 (1) | 4.73 | 4.29 |
| 21M01718-018 | Centrifuge | Apr. 12, 2021 (2) | 7.03 | 6.38 |
| 21M01718-019 | Centrifuge | Apr. 12, 2021 (3) | 5.07 | 4.60 |
| 21M01718-020 | Centrifuge | Apr. 12, 2021 (4) | 4.87 | 4.42 |
| 21M01718-021 | Control | | 4.17 | 3.78 |

### Initial Analysis

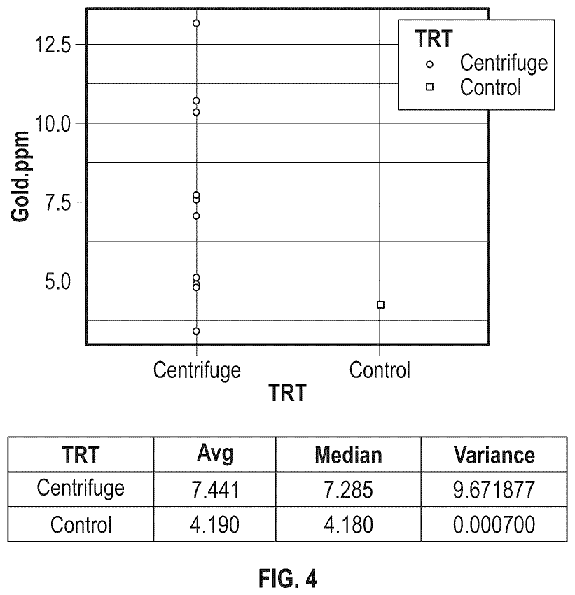

Treated samples measured on average 7.44 ppm Au; a 76% increase from control's 4.19 ppm. The 95% confidence interval of the average sample mean is between 5.512647 ppm at the lower end and 9.367353 ppm at the high end. Control samples average 4.19 ppm. Also, we summarize basic statistical measures. The graph of FIG. 4 depicts treatment versus control results. The table in FIG. 4 shows the average.

### Test Results

From the data in FIG. 4, the conclusion was that the average Au content in the treated sample was statistically significantly higher than control's 4.19 ppm. Moreover, on average, we expect treated samples to yield at least 5.63 ppm of Au, a 33% increase compared to the current industry standard of 4.22 ppm.

### Element and Metals Changes

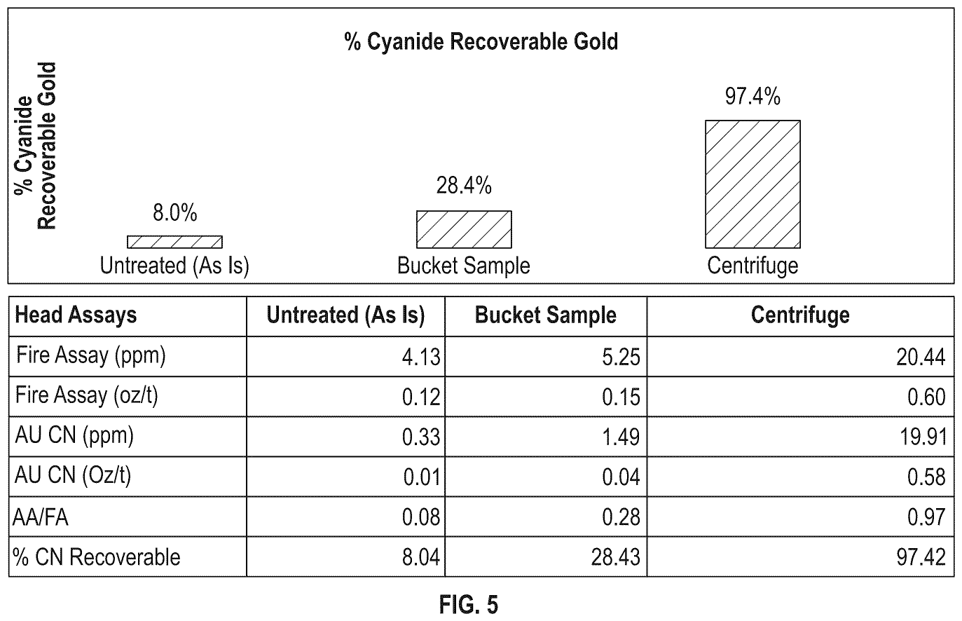

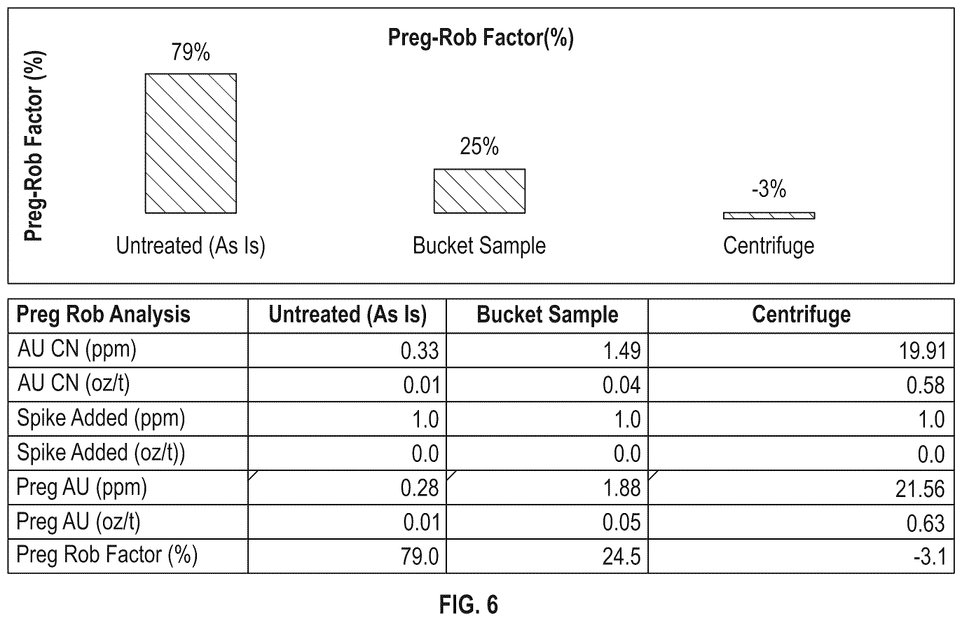

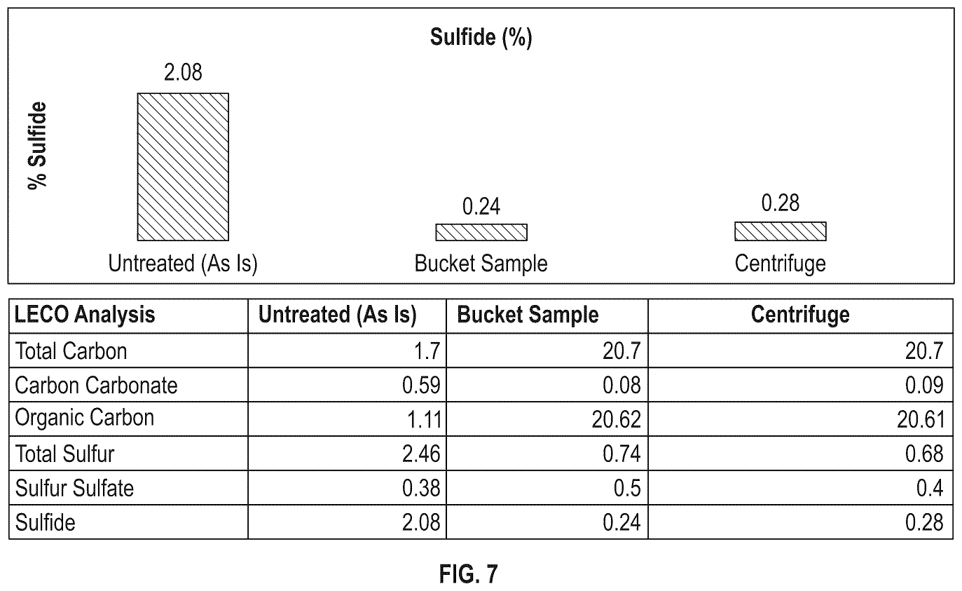

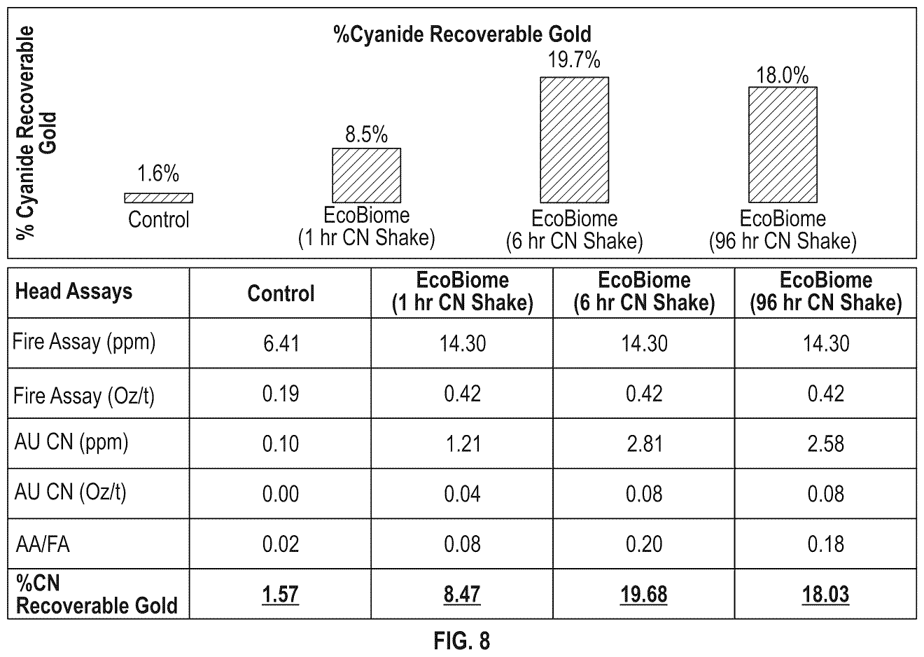

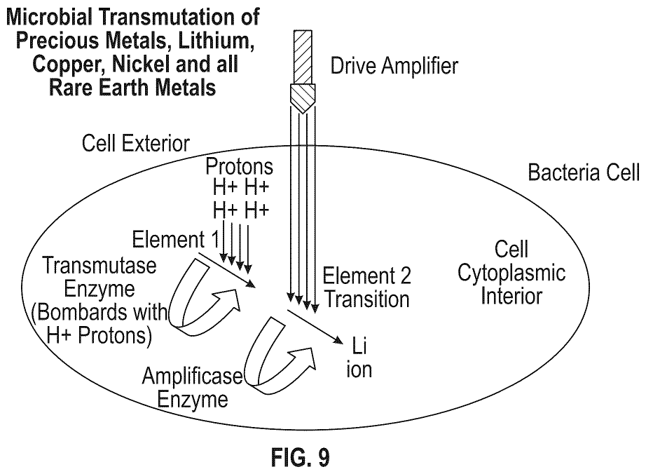

Metals and elements were tracked as a response to changes in Gold concentrations. Table 3 lists average changes in metals content over 5 batch runs, and Table 4 lists rare earth metal concentration changes. Table 5 is a comparison of “Industrial Standard Treatment” using Acidithiobacillus feroxidans vs. ECOAU1 Treated substrates. FIG. 5 presents a bar graph of “% Cyanide Recoverable Gold” and tabular data; FIG. 6 presents a bar graph of “Preg-Rob Factor” with tabular data; FIG. 7 presents a bar graph of % sulfide with tabular data; and FIG. 8 presents a bar graph of “% Cyanide Recoverable Gold” and tabular data at different cyanide shake times.

TABLE 3
Metals and elements tracked as a response
to changes in Gold concentrations

| | Double | EcoBiome | |
|---|---|---|---|
| | Refractory | ‘Cultured GOLD’ | % Change |
| | Ore (ppm; | (ppm; Post | (EcoBiome vs. |
| Elements | As is) | Processing) | As Is Sample) |
| Copper | 89 | 261 | +293% |
| Iron | 18,844 | 14,138 | −25% |
| Magnesium | 12,547 | 0 | −100% |
| Sulfur | 24,043 | 15,060 | −37% |
| Aluminum | 72,742 | 36,747 | −50% |
| Arsenic | 3,516 | 804 | −77% |
| Barium | 484 | 252 | −48% |
| Selenium | 8 | 0 | −100% |
| Nickel | 23 | 0 | −100% |

### 5 Batch Averages

TABLE 4
Rare Earth Element Concentration Changes

| Element | % Increase Over Control (Mean) |
|---|---|
| Neodymium | +53% |
| Lanthanum | +42% |
| Yttrium | +41% |
| Cerium | +54% |
| Dysporium | +65% |
| Praseodymium | +63% |
| Scandium | +61% |
| Lutetium | +59% |

### Method: ICP

TABLE 5
Comparison of Industrial Standard Treatment vs. ECOAU1 Treated

| | | | ECOAU1 |
|---|---|---|---|
| | Untreated | Industrial Standard | Treated |
| | Control | Acidithiobacillus | Thiomonas |
| | Rock Ore As Is | feroxidans | isabelensis |
| MgO | 3.159 | 2.7838 | 1.7399 |
| Al₂O₃ | 17.899 | 10.2713 | 1.6181 |
| Si | 83.347 | 70.2226 | 38.2929 |
| P | 0.0693 | 0.0272 | <LOD |
| S | 2.2189 | 1.9328 | 0.5288 |
| K₂O | 2.7445 | 2.1506 | 0.2892 |
| Ca | 10.993 | 5.1817 | 11.5252 |
| Ti | 2917 | 0.2181 | 0.0432 |
| V | 0.006 | 0.005 | 0.0039 |
| Cr | 0.011 | 0.0099 | 0.0033 |
| Mn | 0.0078 | 0.0054 | 0.012 |
| Fe | 1.9345 | 1.5882 | 0.6074 |
| Co | 0.02101 | 0.0029 | <LOD |
| Ni | 0 | 0 | <LOD |
| Cu | 0.001 | 0.001 | 0.0022 |
| Zn | 0.0093 | 0.0075 | 0.0028 |
| Ga | 0 | 0 | <LOD |
| As | 0.0533 | 0.0364 | 0.0111 |
| Se | 0.0003 | 0.0003 | 0.0002 |
| Rb | 0.0089 | 0.0073 | 0.0017 |
| Sr | 0.0099 | 0.0082 | 0.0117 |
| Y | 0.0069 | 0.0022 | 0.0035 |
| Zr | 0.0344 | 0.0122 | 0.0127 |
| Nb | 0 | 0.003 | <LOD |
| Ru | 0 | 0 | <LOD |
| Rh | 0 | 0 | 0 |
| Pd | 0 | 0 | <LOD |
| In | 0 | 0 | <LOD |
| Sn | 0 | 0 | 0.0059 |
| Sb | 0.045 | 0.015 | 0.0168 |
| Ba | 0.0099 | 0.0093 | 0.0079 |
| La | 0.0089 | 0.0075 | <LOD |
| Hf | 0 | 0.0002 | <LOD |
| Au | 0.159 | 0.6312 | 4.113 |
| TI | 0 | 0.008 | 0.0055 |
| Pb | 0.0009 | 0.0009 | <LOD |
| Th | 0 | 0 | <LOD |

An untreated (“As Is”) sample of gold ore rock was processed through the apparatus known under the trade designation EcoBiome™ Platform to demonstrate changes in the mobility of elemental gold. The standard processing of rock ore was initiated to determine if the addition of the Thiomonas isabelensis microorganism resulted in an increase in gold production and recovery.

The As Is untreated control demonstrated a gold recovery of 8.0% (FIG. 5), whereas the Thiomonas isabelensis treated samples yielded higher and significant improvements in gold recovery and gold production as measured through the Fire Assay (ppm) and CN % recoverable percentages, as shown in Tables 3-5 and FIGS. 5-8. The bucket sample yielded a 28.4% total recovery, and the centrifuge sample yielded a 97.4% increase in recovery of gold from the rock ore sample.

Gold ores associated with carbonaceous matter present several difficulties in their extraction due to the ability of this matter to adsorb the aurocyanide complex from gold pregnant solutions, resulting in low extractions by direct cyanidation. This phenomenon is known as “preg robbing.” To examine the preg rob factor %, standard cyanide processing of rock ore was used to compare against the biotechnology processing using the microorganism Thiomonas isabelensis. The As Is untreated control demonstrated an organic carbon preg robbing factor of 79% (FIG. 6), whereas the Thiomonas isabelensis treated samples yielded lower and significant reductions in preg robbing factors as measured through standard preg robbing tests. The bucket sample yielded a 25.0% total preg robbing factor and the centrifuge sample yielded a −3.0% reg robbing factor or reduction of organic carbon from the rock ore sample.

Moreover, examination was conducted to demonstrate and measure substantial changes in Sulfide % fund in the rock ore (FIG. 7). The As Is untreated control demonstrated a Sulfur Sulfide percentage of 7%, whereas the Thiomonas isabelensis treated samples yielded lower and significant reductions in Sulfur Sulfide percentages as measured through standard LECO and sulfide quantifying assays. The treated bucket sample yielded a 0.24% sulfide % and the treated centrifuge sample yielded a 0.28% sulfide % or reduction of organic carbon from the rock ore sample.

As shown in FIG. 8, the As Is untreated control demonstrated a gold recovery of 1.6%, whereas the EcoBiome Thiomonas isabelensis treated samples yielded higher and significant improvements in gold recovery and gold production as measured through the Fire Assay (ppm) and CN % recoverable percentages. The EcoBiome samples were measured after a 1 hr., 6 hr. and 96 hr. Cyanide Shake test increase in recovery of gold from the rock ore sample. The 1 hr. CN shake test yielded an 8.5% increase (14.30 ppm Fire Assay), the 6 hr. CN shake test yielded a 19.7% increase (14.30 ppm Fire Assay) and the 96 hr. CN shake test yielded a 18.0% increase (14.30 ppm Fire Assay) compared to the untreated control which yielded a 1.6% (6.41 ppm Fire Assay).

An ion selective Lithium probe was used to conduct experimental testing of different substrates and feedstocks containing Lithium sources. Each substrate material was pre-weighed and added to the apparatus known under the trade designation EcoBiome™ Discovery with water and the ECO002 microbe, an isolated modified bacterial strain Glycomyces lithiumensis, which has been designated Accession number NRRL No. B-68190, deposited in accordance with the Budapest Treaty at the Agricultural Research Service Culture Collection (USDA, ARS, 1815 North University Street, Peoria, IL, 61064) on Aug. 15, 2022. All reactions were given a residence time of 2 weeks at 33° C. without agitation. All readings were made in triplicate using an ion selective probe (Lithium specific), and reported in Tables 6, 7, and 8.

TABLE 6
Lithium Source Materials

| | | EcoBiome ECO002 |
|---|---|---|
| Material | Untreated | Treated |
| Lithium batteries (Spent) | 0 ppm | 41 ppm |
| | 0 ppm | 45 ppm |
| | 0 ppm | 54 ppm |
| Spodumene | 0 ppm | 33 ppm |
| (Nascent ore material) | | |
| | 1 ppm | 41 ppm |
| | 0 ppm | 62 ppm |

### Testing by Ion Selective Probe in Solution

TABLE 7
Agriculture Source Materials

| | | EcoBiome ECO002 | |
|---|---|---|---|
| Material | Untreated | Treated | |
| Corn Residue | 0 ppm | 3.8 | ppm |
| (Stalk/Stubble) | | | |
| | 0 ppm | 4.9 | ppm |
| | 0 ppm | 6.4 | ppm |
| Soybean (Stalk/Residue) | 0 ppm | 17.9 | ppm |
| | 0 ppm | 14.3 | ppm |
| | 0 ppm | 18.9 | ppm |

### Testing by Ion Selective Probe in Solution

TABLE 8
Energy/Oil & Gas Source Materials

| | | EcoBiome ECO002 | |
|---|---|---|---|
| Material | Untreated | Treated | |
| Produced/Frac Water | 3 ppm | 95 | ppm |
| | 2 ppm | 107 | ppm |
| | 0 ppm | 122 | ppm |
| Paraffin wax | 0 ppm | 106 | ppm |
| | 0 ppm | 104 | ppm |
| | 0 ppm | 183 | ppm |

### Testing by Ion Selective Probe in Solution

Heavy Metal Bioremediation-Sludge residue originating from power plant waste by-products were treated with specialized heavy metal and sludge remediating microbials. After allowing for microbial bioremediation, both untreated and treated samples were submitted to independent third party laboratories for a full heavy metals analysis. Inductively Coupled Plasma-Atomic Emissions Spectrometry (ICP-AE) tests were performed on heavy metal contaminated sludge in a dry and liquid form. Results are shown in Tables 9, 10, and 11.

TABLE 9
Heavy Metal Assay (Liquid Form; 2018; Untreated
Control vs. EcoBiome Microbial Treated)

| Heavy Metals | Untreated | Microbial Treated* | |
|---|---|---|---|
| (Total) | (mg/kg ppm) | (mg/kg; ppm) | % Change |
| Mercury | n.d. | n.d. | — |
| Zinc | 43.5 | 26.1 | −40% |
| Selenium | 3.08 | 1.14 | −63% |
| Lead | 8.6 | 3.1 | −64% |
| Nickel | 10.2 | 4.2 | −59% |
| Molybdenum | 1.1 | n.d. | −100% |
| Cobalt | 3.31 | 1.54 | −53% |
| Cadmium | 0.65 | n.d. | −100% |
| Arsenic | 4.02 | n.d. | −100% |
| Aluminum | 6.65 | 2.1 | −68% |
| Lithium | 75.75 | n.d. | −100% |

Analytical Methods: EPA 6010B/7471A (Midwest Laboratories, Inc) *Microbial Treatment: Sodium sulfide sludge and carry over residue in a liquid form. Trial duration: 45 days

TABLE 10
Heavy Metal Assay (SOLID Form; 2018; Untreated
Control vs. EcoBiome Microbial Treated)

| Heavy Metals | Untreated | Microbial Treated* | |
|---|---|---|---|
| (Total) | (mg/kg; ppm) | (mg/kg; ppm) | % Change |
| Mercury | 0.02 | n.d. | −100% |
| Barium | 34.8 | 8.12 | −76% |
| Selenium | 6.16 | 1.16 | −81% |
| Lead | n.d. | n.d. | n.d. |
| Silver | n.d. | n.d. | n.d. |
| Chromium | 1.42 | 0.728 | −48% |
| Cadmium | 1.33 | n.d. | −100% |
| Arsenic | 5.67 | 2.50 | −56% |
| Aluminum | 8.99 | 2.23 | −75% |
| Lithium | 45.33 | n.d. | −100% |

Analytical Methods: EPA 6010B/7471A (Chemtech-Ford Laboratories)
*Microbial Treatment: Sodium sulfide sludge and carry over residue in a dry form. Trial Duration: 45 days
*Microbial Treatment: Sludge Pit (Heavy Sludge)

TABLE 11
EcoBiome Microbial Treatment on Different
Ore Types (Silicon to Gold Transmutation)

| | Double | | | Non- |
|---|---|---|---|---|
| | Refractory | Single | Single | Refractory |
| | Ore - High | Refractory | Refractory | Ore - High |
| | Sulfur; High | Ore - High | Ore - High | Arsenic; High |
| Treatments | Carbonaceous | Sulfur | Carbonaceous | Silicon |
| Fire Assay | 0.014 oz/ton | 0.009 oz/ton | 0.006 oz/ton | 0.110 oz/ton |
| CONTROL | | | | |
| Fire Assay | 0.120 oz/ton | 0.114 oz/ton | 0.119 oz/ton | 0.225 oz/ton |
| EcoBiome | | | | |
| Treated | | | | |
| Preg-robbing | 69% | 22% | 81% | 17% |
| Carbon | | | | |
| CONTROL | | | | |
| Preg-robbing | 4% | 2% | 5.5% | 2% |
| Carbon | | | | |
| EcoBiome | | | | |
| Treated | | | | |
| Cyanide | 0% | 6% | 2% | 42% |
| Recoverable Au | | | | |
| CONTROL | | | | |
| Cyanide | >90% | >90% | 88% | >90% |
| Recoverable Au | | | | |
| EcoBiome | | | | |
| Treated | | | | |
| Sulfur Sulfide | 5.30 | 39% | 6.5% | 6% |
| CONTROL | | | | |
| Sulfur Sulfide | 3.10 | 11.5% | 1.8% | 1.2% |
| EcoBiome | | | | |
| Treated | | | | |
| Arsenic | 4.90% | 6.98% | 8.33% | 19.6% |
| Concentration | | | | |
| (%/wt.) | | | | |
| CONTROL | | | | |
| Arsenic (%/wt.) | n.d. | 0.288% | n.d. | n.d. |
| EcoBiome | | | | |
| Treated | | | | |
| Silicon | 19.90% | 41.89% | 32.10% | 58.33% |
| Concentration | | | | |
| (%/wt.) | | | | |
| CONTROL | | | | |
| Silicon (%/wt.) | 3.09% | 10.45% | 4.33% | 8.15% |
| EcoBiome | | | | |
| Treated | | | | |

### 5 Day EcoBiome Microbial Treatment

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All references, patents and patent applications disclosed herein are incorporated by reference with respect to the subject matter for which each is cited, which in some cases may encompass the entirety of the document.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

As used herein, the term “isolated” in the context of an isolated bacterial strain, is one which is altered or removed from the natural state through human intervention.

### SEQUENCE LISTING

### Sequence Listing Information:

DTD Version: V1_3
File Name: 3414.900.xml
Software Name: WIPO Sequence
Software Version: 2.3.0
Production Date: 2023 Sep. 1

### General Information:

Current application/IP Office: US
Current application/Applicant file reference: 3414.900
Earliest priority application/IP Office: US
Earliest priority application/Application number: 63/434,358
Earliest priority application/Filing date: 2022 Dec. 21
Applicant name: Ecobiome Holdings LLC
Applicant name/Language: en
Inventor name: Marc Rodriguez
Inventor name/Language: en
Invention title: Microbial Mediated Transmutations (en)
Sequence Total Quantity: 2

### Sequences:

Sequence Number (ID): 1
Length: 982
Molecule Type: AA
Features Location/Qualifiers:
   source, 1 . . . 982
      >mol_type, protein
      >organism, synthetic construct
Residues:

| | |
|---|---|
| AVQNESKRYT VSYLKTLNYY DLVDLLAKTE IENLPDLFQY | 60 |
| SSDAKEFYGN KTRMNFIMDE | |
| IGRRASQYTE IDHKGIPTLV EVVRAGFYLG FHNKELNEIN | 120 |
| KRSFKERVIP SILAIQKNPN | |
| FKLGTEVQDK IVSATGLLAG NETSPAEVVN NFTPILQDCI | 180 |
| KNMDRYALDD LKSKALFNVL | |
| AAPTYDVTEY LRATKEKPEN TPWYGKIDGF INEVKKLALY | 240 |
| GKINSRNSWI IDNGIYHIAP | |
| LGKLHSNNKI GIETLTEVMK VYPYLSMQHL QSADQIKRHY | 300 |
| DSKDAEGNKI PLDKFKKEGK | |
| EKYCPKTYTF DDGKVIIKAG ARVEEEKVKR LYWASKEVNS | 360 |
| QFFRVYGIDK PLEEGNPDDI | |
| LTMVIYNSPE EYKLNSVLYG YDTNNGGMYI EPEGTFFTYE | 420 |
| REAQESTYTL EELFRHEYTH | |
| YLQGRYAVPG QWGRTKLYDN DRLTWYEEGG AELFAGSTRT | 480 |
| SGILPRKSIV SNIHNTTRNN | |
| RYKLSDTVHS KYGASFEFYN YACMFMDYMY NKDMGILNKL | 540 |
| NDLAKNNDVD GYDNYIRDLS | |
| SNHALNDKYQ DHMQERIDNY ENLTVPFVAD DYLVRHAYKN | 600 |
| PNEIYSEISE VAKLKDAKSE | |
| VKKSQYFSTF TLRGSYTGGV SKGKLEDQKA MNKFIDDSLK | 660 |
| KLDTYSWSGY KTLTAYFTNY | |
| KVDSSNKVTY DVVFHGYLPN EGDSKNSLPY GKTNGTYKGT | 720 |
| EKEKIKFSSE GSFDPDGKIV | |
| SYEWDFGDGN KSNEENPEHS YDKVGTYTVK LKVTDDKGES | 780 |
| SVSTTTAEIR DLSENKLPVI | |
| YMHVPTSGAL NQKVVFYGKG TYDPDGSIAG YQWDFGDGSD | 840 |
| FSSEQNPSHV YTKKGEYTVT | |
| LRVMDSSGQM SEKTMKIKIT DPVYPIGTEK EPNNSKETAS | 900 |
| GPIVPGIPVS GTIENTSDQD | |
| YFYFDVITPG EVKIDINKLG YGGATWVVYD ENNNAVSYAT | 960 |
| DDGQNLSGKF KADKPGRYYI | |
| HLYMENGSYM PYRINIEGSV GR | 982 |

Sequence Number (ID): 2
Length: 982
Molecule Type: AA
Features Location/Qualifiers:
   source, 1 . . . 982
      >mol_type, protein
      >organism, synthetic construct
Residues:

| | |
|---|---|
| AVQNESKRYT VSYLKTLNYY DLVDLLAKTE IENLPDLFQY | 60 |
| SSDAKEFYGN KTRMNFIMDE | |
| IGRRASQYTE IDHKGIPTLV EVVRAGFYLG FHNKELNEIN | 120 |
| KRSFKERVIP SILAIQKNPN | |
| FKLGTEVQDK IVSATGLLAG NETSPAEVVN NFTPILQDCI | 180 |
| KNMDRYALDD LKSKALFNVL | |
| AAPTYDVTEY LRATKEKPEN TPWYGKIDGF INEVKKLALY | 240 |
| GKINSRNSWI IDNGIYHIAP | |
| LGKLHSNNKI GIETLTEVMK VYPYLSMQHL QSADQIKRHY | 300 |
| DSKDAEGNKI PLDKFKKEGK | |
| EKYCPKTYTF DDGKVIIKAG ARVEEEKVKR LYWASKEVNS | 360 |
| QFFRVYGIDK PLEEGNPDDI | |
| LTMVIYNSPE EYKLNSVLYG YDTNNGGMYI EPEGTFFTYE | 420 |
| REAQESTYTL EELFRHEYTH | |
| YLQGRYAVPG QWGRTKLYDN DRLTWYEEGG AELFAGSTRT | 480 |
| SGILPRKSIV SNIHNTTRNN | |
| RYKLSDTVHS KYGASFEFYN YACMFMDYMY NKDMGILNKL | 540 |
| NDLAKNNDVD GYDNYIRDLS | |
| SNHALNDKYQ DHMQERIDNY ENLTVPFVAD DYLVRHAYKN | 600 |
| PNEIYSEISE VAKLKDAKSE | |
| VKKSQYFSTF TLRGSYTGGV SKGKLEDQKA MNKFIDDSLK | 660 |
| KLDTYSWSGY KTLTAYFTNY | |
| KVDSSNKVTY DVVFHGYLPN EGDSKNSLPY GKTNGTYKGT | 720 |
| EKEKIKFSSE GSFDPDGKIV | |
| SYEWDFGDGN KSNEENPEHS YDKVGTYTVK LKVTDDKGES | 780 |
| SVSTTTAEIR DLSENKLPVI | |
| YMHVPTSGAL NQKVVFYGKG TYDPDGSIAG YQWDFGDGSD | 840 |
| FSSEQNPSHV YTKKGEYTVT | |
| LRVMDSSGQM SEKTMKIKIT DPVYPIGTEK EPNNSKETAS | 900 |
| GPIVPGIPVS GTIENTSDQD | |
| YFYFDVITPG EVKIDINKLG YGGATWVVYD ENNNAVSYAT | 960 |
| DDGQNLSGKF KADKPGRYYI | |
| HLYMFNGSYM PYRINIEGSV GR | 982 |
| END | |

What is claimed is:

1. A method of recovering or producing precious metals, platinum group metals, and/or rare earth elements from a starting substrate, the method comprising: contacting the starting substrate with a composition comprising an isolated microbe for an amount of time sufficient for the biological transmutation process to occur, the isolated microbe expressing a first enzyme at least 95% identical to, or at least 98% identical to, or identical to a transmutase enzyme identified by SEQ. ID. NO. 1, and a second enzyme at least 95% identical to, at least 98% identical to, or identical to an amplificase enzyme identified by SEQ. ID. NO. 2.

2. The method of claim 1, wherein the method produces a higher concentration of the rare earth element, Neodymium, and wherein the starting substrate is a rock ore substrate.

3. A method of producing gold from silicon, the method comprising: contacting a starting substrate comprising silicon with composition comprising an isolated microbe for an amount of time sufficient for a biological transmutation process mediated by the microbe to occur, the isolated microbe expressing a first enzyme at least 95% identical to, at least 98% identical to, or identical to a transmutase enzyme identified by SEQ. ID. NO. 1, and a second enzyme at least 95% identical to, at least 98% identical to, or identical to an amplificase enzyme identified by SEQ. ID. NO. 2.

4. The method of claim 3, wherein silicon is transmutated directly into gold by contacting a starting substrate comprising silicon with the microbe.

5. The method of claim 3, wherein, the method bypasses the quartz stage (Silicon Dioxide) through a microbial mediated energy conversion.

6. The method of claim 1 wherein the starting substrate comprises aluminum and the method comprises transmutating at least some of the aluminum into one or more rare earth elements.

7. The method of claim 1, wherein the rare earth element is Neodymium.

8. A method of converting phosphorus in a starting material into phosphoric acid and/or phosphonic acid, the method comprising: contacting a starting substrate comprising phosphorus with composition comprising an isolated microbe for an amount of time sufficient for a biological transmutation process mediated by the microbe to occur, the isolated microbe expressing a first enzyme at least 95% identical to, at least 98% identical to, or identical to a transmutase enzyme identified by SEQ. ID. NO. 1, and a second enzyme at least 95% identical to, at least 98% identical to, or identical to an amplificase enzyme identified by SEQ. ID. NO. 2.

9. A method of converting heavy oil in a starting material into octane, the method comprising: contacting a starting substrate comprising heavy oil with composition comprising an isolated microbe for an amount of time sufficient for a biological transmutation process mediated by the microbe to occur, the isolated microbe expressing a first enzyme at least 95% identical to, at least 98% identical to, or identical to a transmutase enzyme identified by SEQ. ID. NO. 1, and a second enzyme at least 95% identical to, at least 98% identical to, or identical to an amplificase enzyme identified by SEQ. ID. NO. 2.

10. The method of claim 1 wherein the rare earth element is Yttrium.

11. A method of extracting, amplifying, or producing lithium comprising contacting a substrate with a composition comprising an isolated microbe for an amount of time sufficient for a biological transmutation process mediated by the microbe to occur, the isolated microbe expressing a first enzyme at least 95% identical to, at least 98% identical to, or identical to a transmutase enzyme identified by SEQ. ID. NO. 1, and a second enzyme at least 95% identical to, at least 98% identical to, or identical to an amplificase enzyme identified by SEQ. ID. NO. 2.

12. The method of claim 11 wherein the substrate is a geological substrate.

13. The method of claim 11 wherein the substrate is a liquid substrate.

14. The method of claim 12, wherein the substrate is obtained from one or more of a terrestrial, aquatic or marine source.

15. The method of claim 14, wherein, the substrate is one or more of lithium batteries, agricultural crops residue and/or stubble, aluminum, bauxite, spodumene, lithium mine substrate, salt flat, soil, biofilm, sediment, native metal rock and sludge residue.

16. The method of claim 12, wherein the substrate is one or more of sandstone, limestone, shale, coal, chalk deposit formations, refractory rock ore.

17. The method of claim 13, wherein the liquid substrate is obtained from one or more of a oil materials, gas materials, frac water, waste waters, sludge waters, saltwater, freshwater, irrigation systems, ponds, lakes, rivers, and estuaries source.

18. The method of claim 13, wherein an anodic and cathodic LED having a wavelength generator set at a range of 100 Hz-200 KHz is used in the liquid substrate.

19. The method of claim 12 wherein the geological substrate comprises an agriculture crop residue.

20. The method of claim 19, wherein the agriculture crop residue is selected from the group consisting of corn, soybean, rice, cotton stubble and all other crop residue.

21. The method of claim 13 wherein the liquid substrate is selected from an oil, gas or frac water sample.


SEQUENCE LISTING (2 sequences)

SEQ ID NO: 1 - protein, 982 aa, synthetic construct
AVQNESKRYTVSYLKTLNYYDLVDLLAKTEIENLPDLFQYSSDAKEFYGNKTRMNFIMDEIGRRASQYTEIDHKGIPTLVEVVRAGFYLGFHNKELNEINKRSFKERVIPSILAIQKNPNFKLGTEVQDKIVSATGLLAGNETSPAEVVNNFTPILQDCIKNMDRYALDDLKSKALFNVLAAPTYDVTEYLRATKEKPENTPWYGKIDGFINEVKKLALYGKINSRNSWIIDNGIYHIAPLGKLHSNNKIGIETLTEVMKVYPYLSMQHLQSADQIKRHYDSKDAEGNKIPLDKFKKEGKEKYCPKTYTFDDGKVIIKAGARVEEEKVKRLYWASKEVNSQFFRVYGIDKPLEEGNPDDILTMVIYNSPEEYKLNSVLYGYDTNNGGMYIEPEGTFFTYEREAQESTYTLEELFRHEYTHYLQGRYAVPGQWGRTKLYDNDRLTWYEEGGAELFAGSTRTSGILPRKSIVSNIHNTTRNNRYKLSDTVHSKYGASFEFYNYACMFMDYMYNKDMGILNKLNDLAKNNDVDGYDNYIRDLSSNHALNDKYQDHMQERIDNYENLTVPFVADDYLVRHAYKNPNEIYSEISEVAKLKDAKSEVKKSQYFSTFTLRGSYTGGVSKGKLEDQKAMNKFIDDSLKKLDTYSWSGYKTLTAYFTNYKVDSSNKVTYDVVFHGYLPNEGDSKNSLPYGKTNGTYKGTEKEKIKFSSEGSFDPDGKIVSYEWDFGDGNKSNEENPEHSYDKVGTYTVKLKVTDDKGESSVSTTTAEIRDLSENKLPVIYMHVPTSGALNQKVVFYGKGTYDPDGSIAGYQWDFGDGSDFSSEQNPSHVYTKKGEYTVTLRVMDSSGQMSEKTMKIKITDPVYPIGTEKEPNNSKETASGPIVPGIPVSGTIENTSDQDYFYFDVITPGEVKIDINKLGYGGATWVVYDENNNAVSYATDDGQNLSGKFKADKPGRYYIHLYMFNGSYMPYRINIEGSVGR

SEQ ID NO: 2 - protein, 982 aa, synthetic construct; identical to SEQ ID NO: 1